(19) United States
(12) Patent Application Publication
(10) Pub. No.: US 2018/0325762 A1
(43) Pub. Date: Nov. 15, 2018

(54) MODULAR ELECTRONIC LOCKING DRAWER SYSTEM AND METHOD

(71) Applicant: Ergotron, Inc., St. Paul, MN (US)

(72) Inventor: Nicholas Simon Hazzard, Minneapolis, MN (US)

(21) Appl. No.: 15/529,000

(22) PCT Filed: Nov. 17, 2015
(86) PCT No.: PCT/US2015/061077

§ 371 Date: May 23, 2017

Related U.S. Application Data
(60) Provisional application No. 62/084,052, filed on Nov. 25, 2014.

(51) Int. Cl. A61G 12/00; G07C 9/00; A61B 50/13; G06F 19/00; E05B 65/46

(52) U.S. Cl. CPC A61G 12/001; G07C 9/00896; A47B 2031/006; G06F 19/00; E05B 65/46; A61B 50/13

(57) ABSTRACT

A drawer system including a structure to receive a plurality of drawer modules individual ones of the drawer modules including a drawer processor, a drawer memory to store a drawer module address, and a latch mechanism to lock one or more drawers in a drawer module. The system including a controller circuit to control access to the drawer modules based on user input, the controller circuit including a controller circuit processor, a controller circuit memory, and a plurality of communication channels daisy-chained such that a first one of the plurality of communication channels is connected between the controller circuit and a drawer processor of a first one of the drawer modules, and a second one of the plurality of communication channels is connected between the drawer processor of the first one of the drawer modules and a drawer processor of a second one of the drawer modules.

### CLAIM OF PRIORITY

This patent application claims the benefit of priority to U.S. Provisional Patent Application Ser. No. 62/084,052 to Hazzard, titled “MODULAR ELECTRONIC LOCKING DRAWER SYSTEM” and filed on Nov. 25, 2014 (Attorney Docket No. 5983.267PRV), which is hereby incorporated by reference herein in its entirety.

### BACKGROUND

With the advent of electronic medical records there has been a need for a mobile platform to facilitate easy transport and/or battery backup of computer systems that medical personnel can use to interface with patients. With the addition of locking drawers to these mobile platforms, commonly known as computer on wheels (COWs), it allows for secure delivery of basic medication. Often one drawer may be tied to a particular patient or type of medication thus the need for multiple discrete drawers becomes apparent. Existing systems are limited to a specific number of drawers and are not easily reconfigurable.

### SUMMARY

The present inventors have recognized a need for a modular electronic locking drawer system that is easily expandable to include more drawers. The electronic locking drawer system can include a main controller circuit, an input device, and a plurality of drawer modules where the circuitry of each drawer module is identical and each drawer module can be controlled or addressed individually.

To further illustrate the MODULAR ELECTRONIC LOCKING DRAWER SYSTEM and METHOD disclosed herein, a non-limiting list of examples is provided here:

In Example 1, a drawer system cart comprise: a structure to receive a plurality of N drawer modules, where N is an integer, individual ones of the plurality of N drawer modules including: a drawer processor; a drawer memory to store a drawer module address; and a latch mechanism to lock one or more drawers in a drawer module; an input device to receive user input; a controller circuit at the structure and in communication with the input device to control access to the plurality of N drawer modules based on the received user input. The controller circuit can include: a controller circuit processor; a controller circuit memory; and a plurality of communication channels daisy-chained such that: a first one of the plurality of comtnunication channels is connected between the controller circuit and a drawer processor of a first one of the plurality of N drawer modules, and a second one of the plurality of communication channels is connected between the drawer processor of the first one of the plurality of N drawer modules and a drawer processor of a second one of the plurality of N drawer modules.

In Example 2, the drawer system of Example 1 can optionally be configured such that the plurality of communication channels are daisy-chained such that: an (N−1) th one of the plurality of communication channels is connected between the drawer processor of the (N−2)th one of the plurality of N drawer modules and a drawer processor of a (N−1)th one of the plurality of N drawer modules, and an Nth one of the plurality of communication channels is connected between the drawer processor of the (N−1)th one of the plurality of N drawer modules and a drawer processor of an Nth one of the plurality of N drawer modules,

In Example 3, the drawer system of any one or any combination of Examples 1-2 can optionally be configured such that the controller circuit processor is configured to assign a first address to the first one of the plurality of N drawer modules, wherein the drawer processor of the first one of the plurality of N drawer modules is configured to assign a second address to the second one of the plurality of N drawer modules, and wherein the drawer processor of the second one of the plurality of N drawer modules is configured to assign a third address to the third one of the plurality of N drawer modules.

In Example 4, the drawer system of Example 3 can optionally be configured such that the drawer processor of the first one of the plurality of N drawer modules configured to assign a second address to the second one of the plurality of N drawer modules is configured to: increment the first address of the first one of the plurality of N drawer modules; and assign the incremented first address as the second address to the second one of the plurality of N drawer modules.

In Example 5, the drawer system of any one or any combination of Examples 1-4 can optionally be configured such that the controller circuit is configured to receive a plurality of drawer module addresses, and wherein the controller circuit memory is configured to store the received plurality of drawer module addresses.

In Example 6, the drawer system of any one or any combination of Examples 1-5 can optionally he configured such that the drawer memory and the controller circuit memory are volatile memory.

In Example 7, the drawer system of any one or any combination of Examples 1-6 can optionally be configured such that the controller circuit processor and each drawer processor of the plurality of N drawer modules communicate using a serial communication protocol.

In Example 8, a machine-implemented method of assigning a respective, unique address to a plurality of drawer modules in an electronic drawer system, the method can comprise: generating, using a controller circuit processor, a first address for a first drawer module; transmitting, using the controller circuit processor, the first address for the first drawer module; receiving, using a drawer processor of the first drawer module, the first address; storing the first address in a drawer memory of the first drawer module; generating, using the drawer processor of the first drawer module, a second address for a second drawer module; transmitting, using the drawer processor of the first drawer module, the second address for the second drawer module; receiving, using a drawer processor of the second drawer module, the second address; and storing the second address in a drawer memory of the second drawer module.

In Example 9, the method of Example 8 can optionally be configured to further comprise: transmitting, by the drawer processor of the second drawer module, the second address; receiving, by the drawer processor of the first drawer module, the second address; transmitting, by the drawer processor of the first drawer module, the second address; receiving, by the controller circuit processor, the second address; and storing the second address in a controller circuit memory.

In Example 10, the method of any one or any combination of Examples 8-9 can optionally be configured to further comprise: generating, by the drawer processor of the second drawer module, a third address for a third drawer module; transmitting, by the drawerprocessor of the second drawer module, the third address for the third drawer module; waiting, by the drawer processor of the second drawer module, for an acknowledgement generated by the third drawer module, wherein the acknowledgement includes the third address; and upon receiving the acknowledgment, transmitting, by the drawer processor of the second drawer module, the second address to the drawer processor of the first drawer module.

In Example 11, the method of any one or any combination of Examples 8-10 can optionally be configured such that the generating, the transmitting, and the receiving of the first address and the second address occur each time power is applied to the electronic drawer system.

In Example 12, the method of any one or any combination of Examples 10-11 can optionally be configured to further comprise receiving, by the controller circuit processor, a drawer module address received using an input device; transmitting, by the controller circuit processor, the drawer module address; receiving, by the drawer processor of the first drawer module, the drawer module address; comparing, by the drawer processor of the first drawer module, the drawer module address and the first address; and controlling a latch mechanism of the first drawer module to operate in response to the drawer module address matching the first address during the comparison.

In Example 13, the method of Example 12 can optionally be configured to further comprise: in response to the drawer module address not matching the first address during the comparison, transmitting, by the drawer processor of the first drawer module, the drawer module address to the drawer processor of the second drawer module; comparing, by the drawer processor of the second drawer module, the drawer module address and the second address; and controlling a latch mechanism of the second drawer module to operate in response to the drawer module address matching the second address during the comparison.

In Example 14, the method of any one or any combination of Examples 10-13 can optionally he configured to further comprise receiving, by the controller circuit processor, a drawer module address received using an input device; transmitting, by the controller circuit processor, the drawer module address; receiving, by the drawer processor of the first drawer module, the drawer module address; comparing, by the drawer processor of the first drawer module, the drawer module address and the first address; and programming a latch mechanism of the first drawer module to operate in response to the drawer module address matching the first address during the comparison.

In Example 15, a drawer system can comprise: a structure to receive a plurality of N drawer modules, where N is an integer, individual ones of the plurality of N drawer modules including: a drawer processor; a drawer memory to store a drawer module address; and a plurality of latch mechanisms to lock two or more drawers in a drawer module; an input device to receive user input; a controller circuit at the structure and in communication with the input device to control access to the plurality of N drawer modules based on the received user input, the controller circuit including: a controller circuit processor; a controller circuit memory; and a plurality of communication channels daisy-chained such that: a first one of the plurality of cotnmunication channels is connected between the controller circuit and a drawer processor of a first one of the plurality of N drawer modules, and a second one of the plurality of communication channels is connected between the drawer processor of the first one of the plurality of N drawer modules and a drawer processor of a second one of the plurality of N drawer modules.

In Example 16, the drawer system or method of any one or any combination of Examples 1-15 can optionally be configured such that all elements, operations, or other options recited are available to use or select from.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an example of a modular electronic locking drawer system that can he used to implement various techniques of this disclosure.

FIG. 2 is a block diagram depicting example components of the modular electronic locking drawer system.

FIG. 3 is a flow diagram depicting an example of a method for assigning a respective, unique address to a plurality of drawer modules in an electronic drawer system.

FIG. 4 is a perspective view of another example of a modular electronic locking drawer system that can be used to implement various techniques of this disclosure.

FIG. 5 is a perspective view of another example of a modular electronic locking drawer system that can be used to implement various techniques of this disclosure.

### DETAILED DESCRIPTION

This disclosure describes a modular electronic locking drawer system that is easily expandable to include more drawers. The electronic locking drawer system can include a main controller circuit, an input device, and a plurality of drawer modules where the circuitry of each drawer module is identical and each drawer module can he controlled or addressed individually. As described in detail below, the locking drawer system can utilize a plurality of communication channels configured in a daisy-chain arrangement such that information is relayed from the main controller circuit through a drawer processor of any intervening drawer module until the information reaches its destination.

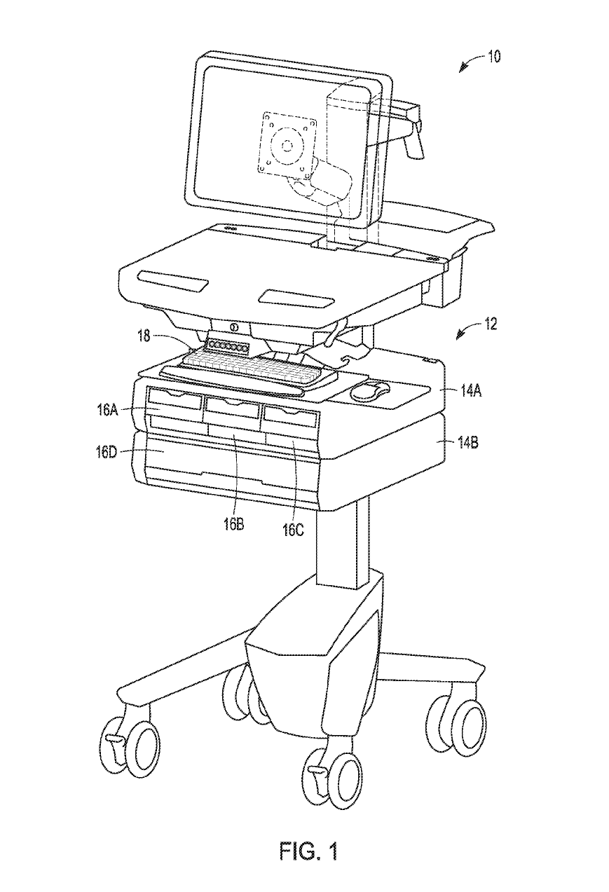

FIG. 1 is a perspectiveperspectiveview of an example of a modular electronic locking drawer system that can be used to implement various techniques of this disclosure. The example of a locking drawer system 10 of FIG. 1 can include a structure, shown generally at 12, to receive one or more drawer modules 14A, 14B (referred to in this disclosure as “drawer modules 14”). In some examples, the structure 12 can be a frame, or a portion of a frame, e.g., a riser.

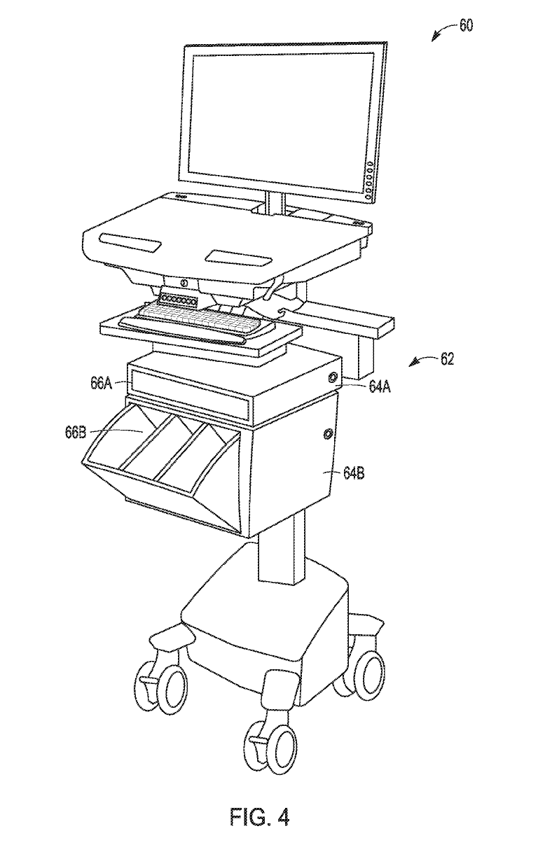

Each drawer module 14 can include one or more drawer housings 16A, 16B, 16C, 16D (referred to in this disclosure as “drawer housings 16”). For example, the drawer module 14A can include three drawer housings 16A-16C and the drawer module 14B can include one drawer housing 16D. In some ex.ample configurations, the drawer housings can slide in and out horizontally. In another example, such as shown and described below with respect to FIG. 4, the drawer housings can pivot about an axis into an open or closed position.

The drawer system 10 can include a user input device 18, e.g., a keyboard or other keypad entry device, a mouse, a touchscreen, and the like, for receiving codes, commands, or other user input from a user to control and/or program operation of the drawer modules 14.

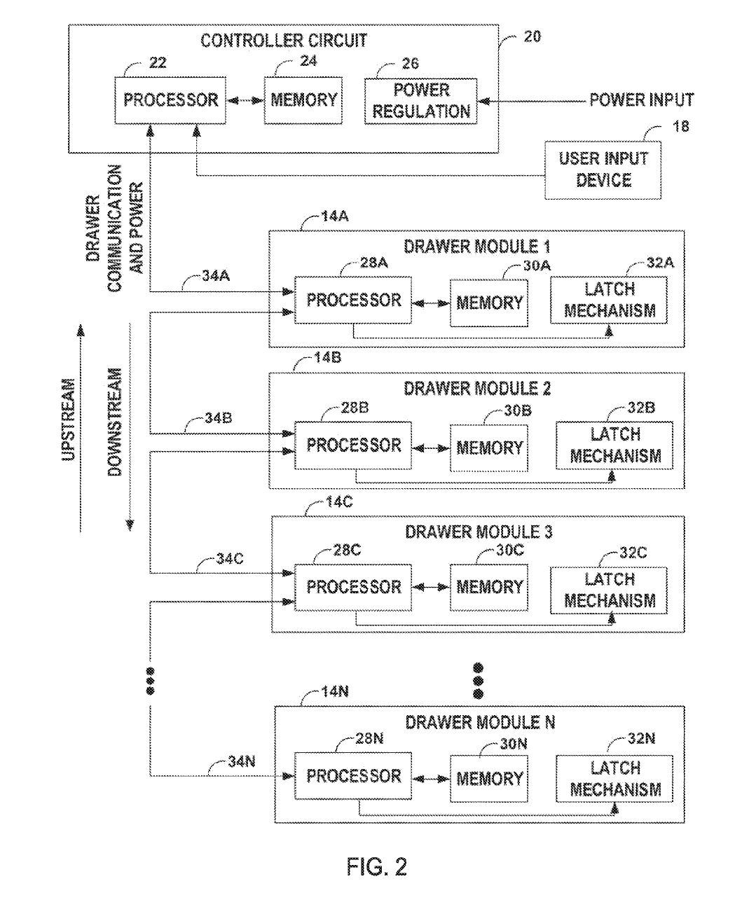

FIG. 2 is a block diagram depicting example components of the modular electronic locking drawer system. The system 10 of FIG. 1 can include a controller circuit 20 having a processor 22, a memory circuit 24, and a power regulation circuit 26 to receive power input, e.g., from a battery or an electrical outlet. The controller circuit 20 can be in communication with the user input device 18 to control access to the plurality of drawer modules 14 based on received user input. In some example configurations, the controller circuit 20 can be located at the structure 12 (FIG. 1) of the system.

The system 10 of FIG. I can include a plurality of N drawer modules 14A-14N. In some examples, individual ones of the drawer modules 14A-14N can include a processor 28, a memory 30, and a latch mechanism 32 to lock one or more drawers in a drawer module. The latch mechanism 32 of a drawer module 14 can control, e.g., lock/unlock, one or more drawer housings. For example, a latch mechanism 32A of drawer module 14A of FIG. 1 can unlock one or more of drawer housings 16A-16C. In another example configuration, a drawer module, e.g., drawer module 14A, can include two or more latch mechanisms to control, e.g., lock unlock, two or more drawer housings, e.g., drawer housings 16A-16C.

The processor 22 of the controller circuit 20 can receive user input from the user input device 18 and transmit signals representing the user input to an addressed drawer module 14.

The system 10 of FIG. 1 can include a plurality of communication channels 34A-34N (referred to in this disclosure as “communication channels 34”). The plurality of communication channels 34 can be daisy-chained such that a first one of the plurality of communication channels, e.g., channel 34A, is connected between the controller circuit 20 and a drawer processor 28 of a first one of the plurality of N drawer modules, and a second one of the plurality of communication channels, e.g., channel 34B, is connected between the drawer processor 28 of the first one of the plurality of N drawer modules 14 and a drawer processor 28 of a second one of the plurality of N drawer modules 14.

In some example configurations that include more than two drawer modules 14, such as shown in FIG. 2, the plurality of communication channels 34 can be further daisy-chained such that an (N−1)th one of the plurality of communication channels 34 is connected between the drawer processor of the (N−2)th one of the plurality of N drawer modules and a drawer processor of a (N−1)th one of the plurality of N drawer modules, and an Nth one of the plurality of communication channels is connected between the drawer processor of the (N−1)th one of the plurality of N drawer modules and a drawer processor of an Nth one of the plurality of N drawer modules.

The controller circuit 20 is communication with the user input device 18 and one drawer module, e.g., drawer module 14A of FIG. 2. If an additional drawer module is desired, e.g., drawer module 14B, it can be connected to the existing drawer module, e.g., drawer module 14A. The additional drawer module 14B, and in particular, the processor 28B of the drawer module 14B, communicates directly to the existing drawer module 14A, and in particular, the processor 14A of the drawer module 14A, and not to the controller circuit 20.

Another drawer module, e.g., drawer module 14C, can be connected to the drawer module 14B. The additional drawer module 14C, a.nd in particular, the processor 14C of the drawer module 14C, can communicate directly to the drawer module 14B, and in particular, the processor 28B of the drawer module 14B, and not to the controller circuit 20. Any number of drawer modules 14 can be added in this manner, depending upon the power limitations of the system 10 and the number of unique addresses available.

The configuration of FIG. 2 can allow each drawer module 14 to be assigned a communication address based on the order in which it is connected to the system 10. In one example implementation, the processor 22 of the controller circuit 20 can assign and transmit an address of “1” to the drawer module 14 to which it is connected, e.g., drawer module 14A in FIG. 2, via the communication channel 34A. The processor 28 of this drawer module 14, e.g., processor 28A, receives and stores in the memory 30, the address “1”.

In addition, the processor 28 of this drawer module 14, processor 28A, can increment its address to generate a new address and attempt to assign the new address, e.g., an address of “2”, to another drawer module. For example, the processor 28A can attempt to transmit the new address, e.g., address “2”, using the communication channel 34B, to the processor 28B of the drawer module 14B. The processor 14A can wait for an acknowledgement signal generated and transmitted by the processor 28B of the drawer module 14B, where the acknowledgement includes the address “2”.

If the drawer module 14B is connected, the processor 28B receives and stores in the memory 30B, the address “2”, and transmits an acknowledgement signal back to the processor 28A. In one example, the acknowledgement signal includes the address, e.g., address “2”, which the processor 28A relays to the processor 22 of the controller circuit 20. Upon receiving the acknowledgment, the processor 28A can transmit the address “2” to the controller circuit 20.

This address assignment process can continue until no further drawer modules 14 are connected. As each drawer module 14 is assigned an address, the processor 28 of that drawer module can transmit information toward the controller circuit 20 (upstream communication) to the drawer module to which the transmitting drawer module is connected. This upstream communication can be relayed through all of the interconnected drawer modules 14 and, in particular, through their respective processors 28 using the plurality of communication channels 34, until the communication reaches the controller circuit 20. The processor 22 of the controller circuit 20 call store in the memory 24 information that identifies the addresses of each drawer in the system.

In one example, this address assignment process can take place automatically when the system 10 receives power. In another example, the address assignment process can be initiated by a unique command entered into the keypad by the user. In some examples, the memory 30 of the drawer modules can be volatile memory such that the assigned addresses are stored when power is applied to the system 10.

After each drawer module 14 is assigned an address, the processor 22 of the controller circuit 20 can transmit instructions to control the latch mechanism 32 of a. particular drawer module, e.g., a drawer unlock or lock instruction. In some examples, the processor 22 of the controller circuit 20 can transmit instructions to program the latch mechanism 32 of a particular drawer module. For example, the programming instructions can include a time interval after which the latch mechanism 32 can automatically relook all unlock drawer module, e.g., 1-30 seconds.

A user can enter a command using the user input device 18, e.g., keypad, to control or program, for example, a drawer module, e.g., drawer module 14B. The processor 22 of the controller circuit 20 can receive the command and transmit the instruction and its associated address to the drawer module with address “1”, e.g., drawer module 14A, using the communication channel 34A. The processor of the drawer module with address “1”, e.g., processor 28A of the drawer module 14A, can compare its stored address with the received address.

If the received address does not match the address of the drawer module, e.g., address “1”, the processor of the drawer module can transmit the instruction and its associated address to the drawer module with address “2” to which the transmitting drawer module is connected (downstream communication). The instruction and address are relayed downstream through each drawer module 14 until the address reaches the drawer module with the unique address contained in the command. Then, the processor 28 of that drawer module executes instructions to perform the requested command.

Similarly, in some examples, an event generated by a drawer module, e.g., drawer module 14N, can cause the processor 28 of the drawer module to transmit the event information along with the address of the drawer module upstream through each connected drawer module 14 until it reaches the controller circuit 20. In this manner, the event information, e.g., open/closed status, error status, latch locked, latch unlocked, latch error, drawer configuration, drawer configuration unknown, resend, and the like, and the address of the drawer module can be relayed through the plurality of communication channels 34 to the controller circuit 20.

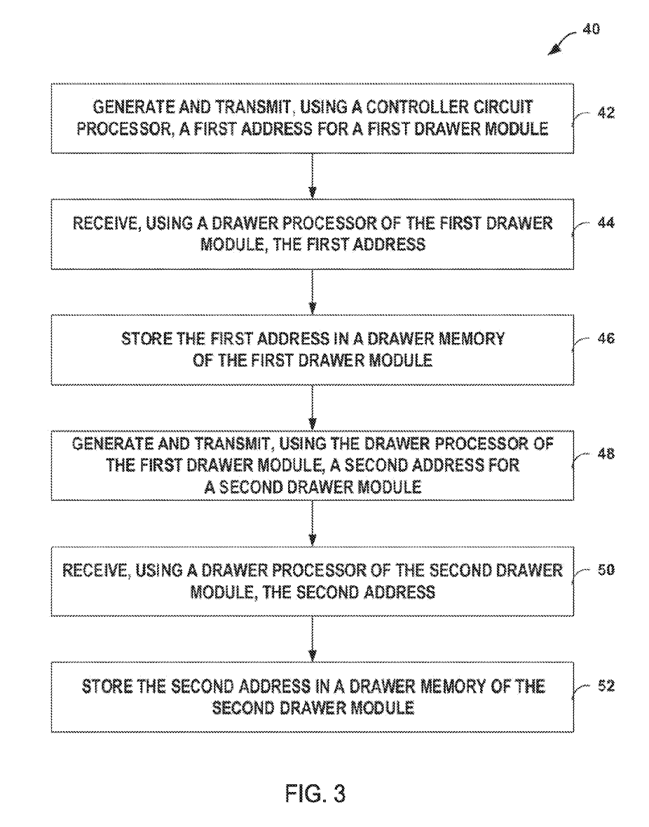

FIG. 3 is a flow diagram depicting an example of a method 40 for assigning a respective, unique address to a plurality of drawer modules in an electronic drawer system. The method 40 can include a processor of a controller circuit, e.g., processor 22 of the controller circuit 20, that can generate a first address for a first drawer module, e.g., drawer module 14A, (block 42), and then can transmit the first address for the first drawer module, e.g., using first communication channel 34A. The drawer processor of the first drawer module, e.g., the drawer processor 27A of the drawer module 14A, can receive the first address (block 44) and store the first address in a drawer memory of the first drawer module, e.g., the drawer memory 30A of the drawer module 14A (block 46). The drawer processor of the first drawer module, e.g,, the drawer processor 28A of the drawer module 14A, can generate and transmit a second address for a second drawer module, e.g., the drawer module 149 (block 48). A drawer processor of the second drawer module, e.g., the drawer processor 289 of the drawer module 14B, can receive the second address (block 50) and store the second address in a drawer memory of the second drawer module, e.g., the drawer memory 30B (block 52).

After receiving the second address, the drawer processor of the second drawer module, e.g., the drawer processor 28B of the drawer module 14B, can transmit the second address to the drawer processor of the first drawer module, The drawer processor of the first drawer module can receive the second address and transmit it to controller circuit. The controller circuit processor 22 can receive and store the second address in the controller circuit memory 24,

The drawer processor of the second drawer module can generate a. third address for a third drawer module and transmit the third address for the third drawer module, e.g., communication channel 34C. The drawer processor of the second drawer module can wait for an acknowledgement signal generated by the third drawer module. In some examples, the acknowledgement signal can include the third address.

Upon receiving the acknowledgment, the drawer processor of the second drawer module can transmit the second address to the drawer processor of the first drawer module. The drawer processor the first drawer module, in turn, can receive the third address and transmit it to controller circuit. The controller circuit processor 22 can receive and store the third address in the controller circuit memory 24. This process of assignng addresses downstream to the drawer modules and relaying the addresses upstream to the controller circuit for storing can continue for all N drawer modules.

In some examples, the generating, transmitting, and receiving of the first, second, etc. addresses occur each time power is applied to the electronic drawer system 10.

To unlock or lock a drawer, a user cart input an address of a drawer module or a code representing an address of a drawer module, for example, using the user input device, e.g., a keypad, keyboard, touchscreen and the like. The controller circuit processor 22 can receive the code representing the drawer module address received using an input device. The controller circuit processor can transmit the drawer module address. The drawer processor of the first drawer module can receive the drawer module address, compare the drawer module address and the first address, and control the latch mechanism of the first drawer module to operate, e.g., lock/unlock, in response to the drawer module address matching the first address during the comparison. In response to the drawer module address not matching the first address during the comparison, the drawer processor of the first drawer module can transmit the drawer module address to the drawer processor of the second drawer module. The drawer processor of the second drawer module can compare the drawer module address and the second address, and control a latch mechanism of the second drawer module to operate in response to the drawer module address matching the second address during the comparison.

FIG. 4 is a perspective view of another example of a modular electronic locking drawer system that can be used to implement various techniques of this disclosure. The example of a locking drawer system 60 of FIG. 4 can include a structure, shown generally at 62, to receive one or more drawer modules 64A, 64B (referred to in this disclosure as drawer modules 64).

Each drawer module 64 can include one or more drawer housings 66A, 66B, (referred to in this disclosure as “drawer housings 66”). The drawer system 60 can include a user input device (not depicted), e.g., a keyboard or other keypad entry device, a mouse, a touchscreen, and the like, for receiving codes, commands, or other user input from a user to control and/or program operation of the drawer modules 64.

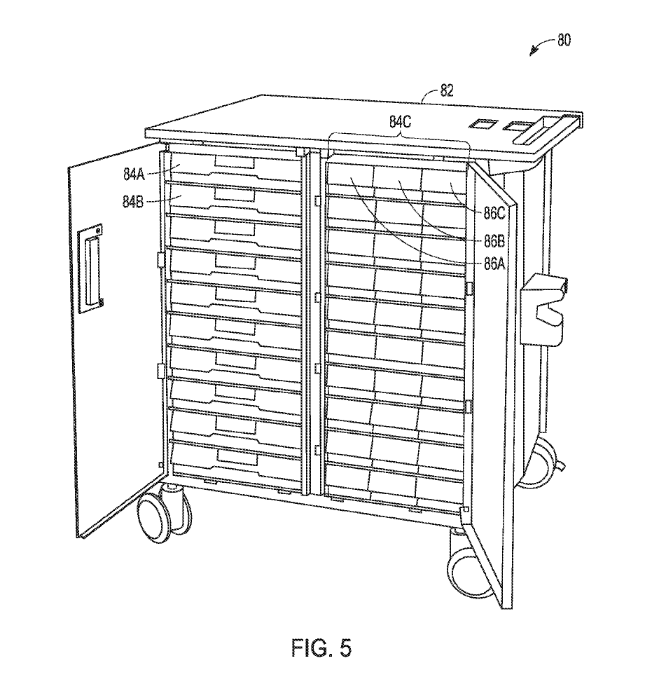

FIG. 5 is a perspective view of another example of a modular electronic locking drawer system that can be used to implement various techniques of this disclosure. The example of a locking drawer system 80 of FIG. 5 can include a structure 82, to receive one or more drawer modules 84A, 84B, and 84C (referred to in this disclosure as drawer modules 84).

Each drawer module 84 can include one or more drawer housings 86A, 86B, and 86C (referred to in this disclosure as “drawer housings 86”. The drawer system 80 can include a user input device (not depicted), e.g., a keyboard or other keypad entry device, a mouse, a touchscreen, and the like, for receiving codes, commands, or other user input from a user to control and/or program operation of the drawer modules 84.

### Additional Notes

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples,” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” in this document, the term “or” is used to refer to a nonexclusive or, such that “A or B” includes “A but not B,” “B but not: ,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with time full scope of equivalents to which such claims are entitled.

1. A drawer system comprising:
a structure to receive a plurality of N drawer modules, where N is an integer, individual ones of the plurality of N drawer modules including:
a drawer processor;
a drawer memory to store a drawer module address; and
a latch mechanism to lock one or more drawers in a drawer module;
an input device to receive user input;
a controller circuit at the structure and in communication with the input device to control access to the plurality of N drawer modules based on the received user input, the controller circuit including:
a controller circuit processor. wherein the controller circuit processor is configured to generate a first address and assign the first address to a first one of the plurality of N drawer modules,
wherein the drawer processor of the first one of the plurality of N drawer modules is configured to generate a second address and assign the second address to a second one of the plurality of N drawer modules, wherein the drawer processor of the second one of the plurality of N drawer modules is configured to generate a third address;
a controller circuit memory; and
a plurality of communication channels daisy-chained such that:
a first one of the plurality of communication channels is connected between the controller circuit and a drawer processor of a first one of the plurality of N drawer modules, and
a second one of the plurality of communication channels is connected between the drawer processor of the first one of the plurality of N drawer modules and a drawer processor of a second one of the plurality of N drawer modules.

2. The drawer system of claim 1, wherein N is an integer greater than 2, wherein the plurality of communication channels are electronically connected such that:
an (N−1)th one of the plurality of communication channels is connected between the drawer processor of the (N−2)th one of the plurality of N drawer modules and a drawer processor of a (N−1)th one of the plurality of N drawer modules, and
an Nth one of the plurality of communication channels is connected between the drawer processor of the (N−1)th one of the plurality of N drawer modules and a drawer processor of an Nth one of the plurality of N drawer modules.

3. The drawer system of claim 1,
wherein the drawer processor of the second one of the plurality of N drawer modules is configured to assign a third address to the third one of the plurality of N drawer modules.

4. The drawer system of claim 1, wherein the drawer processor of the first one of the plurality of N drawer modules configured to assign a second address to the second one of the plurality of N drawer modules is configured to:
increment the first address of the first one of the plurality of N drawer modules; and
assign the incremented first address as the second address to the second one of the plurality of N drawer modules.

5. The drawer system of any one of claim1, wherein the controller circuit is configured to receive a plurality of drawer module addresses, and wherein the controller circuit memory is configured to store the received plurality of drawer module addresses.

6. The drawer system of any one of claim 1, wherein the drawer memory and the controller circuit memory are volatile memory.

7. The drawer system of any one of claim 1, wherein the controller circuit processor and each drawer processor of the plurality of N drawer modules communicate using a serial communication protocol.

8. A machine-implemented method of assigning a respective, unique address to a plurality of drawer modules in an electronic drawer system, the method comprising:
generating, using a controller circuit processor, a first address for a first drawer module;
transmitting, using the controller circuit processor, the first address for the first drawer module;
receiving, using a drawer processor of the first drawer module, the first address;
storing the first address in a drawer memory of the first drawer module;
generating, using the drawer processor of the first drawer module, a second address for a second drawer module;
transmitting, using the drawer processor of the first drawer module, the second address for the second drawer module;
receiving, using a drawer processor of the second drawer module, the second address; and
storing the second address in a drawer memory of the second drawer module.

9. The method of claim 8, comprising:
transmitting, by the drawer processor of the second drawer module, the second address;
receiving, by the drawer processor of the first drawer module, the second address;
transmitting, by the drawer processor of the first drawer module, the second address;
receiving, by the controller circuit processor, the second address; and
storing the second address in a controller circuit memory.

10. The method of claim 8, comprising:
generating, by the drawer processor of the second drawer module, a third address for a third drawer module;
transmitting, by the drawer processor of the second drawer module, the third address for the third drawer module;
waiting, by the drawer processor of the second drawer module, for an acknowledgement generated by the third drawer module, wherein the acknowledgement includes the third address; and
upon receiving the acknowledgment, transmitting, by the drawer processor of the second drawer module, the second address to the drawer processor of the first drawer module.

11. The method of claim 8, wherein the generating, the transmitting, and the receiving of the first address and the second address occur each time power is applied to the electronic drawer system.

12. The method of claim 10, comprising:
receiving, by the controller circuit processor_{;} a drawer module address received using an input device;
transmitting, by the controller circuit processor, the drawer module address;
receiving, by the drawer processor of the first drawer module, the drawer module address;
comparing, by the drawer processor of the first drawer module_{;} the drawer module address and the first address; and
controlling a latch mechanism of the first drawer module to operate in response to the drawer module address matching the first address during the comparison.

13. The method of claim 12, comprising:
in response to the drawer module address not matching the first address during the comparison, transmitting, by the drawer processor of the first drawer module, the drawer module address to the drawer processor of the second drawer module;
comparing, by the drawer processor of the second drawer module, the drawer module address and the second address; and
controlling a latch mechanism of the second drawer module to operate in response to the drawer module address matching the second address during the comparison.

14. The method of claim 10, comprising:
receiving, by the controller circuit processor, a drawer module address received using an input device;
transmitting, by the controller circuit processor, the drawer module address;
receiving, by the drawer processor of the first drawer module, the drawer module address;
comparing, by the drawer processor of the first drawer module, the drawer module address and the first address; and
programming a latch mechanism of the first drawer module to operate in response to the drawer module address matching the first address during the comparison.

15. A drawer system comprising:
a structure to receive a plurality of N drawer modules, where N is an integer, individual ones of the plurality of N drawer modules including:
a drawer processor;
a drawer memory to store a drawer module address; and
a plurality of latch mechanisms to lock two or more drawers in a drawer module;
an input device to receive user input;
a controller circuit at the structure and in communication with the input device to control access to the plurality of N drawer modules based on the received user input, the controller circuit including:
a controller circuit processor; wherein the controller circuit processor is configured to generate a first address and assign the first address to a first one of the plurality of N drawer modules,
wherein the drawer processor of the first one of the plurality of N drawer modules is configured to generate a second address and assign the second address to a second one of the plurality of N drawer modules, wherein the drawer processor of a second one of the plurality of N drawer modules is configured to generate a third address;
a controller circuit memory; and
a plurality of communication channels electrically connected such that:
a first one of the plurality of communication channels is connected between the controller circuit and a drawer processor of a first one of the plurality of N drawer modules, and
a second one of the plurality of communication channels is connected between the drawer processor of the first one of the plurality of N drawer modules and a drawer processor of a second one of the plurality of N drawer modules.

16. The drawer system of claim 15, wherein N is an integer greater than 2, wherein the plurality of communication channels are electrically connected such that:
an (N−1)th one of the plurality of communication channels is electrically connected between the drawer processor of the (N−2)th one of the plurality of N drawer modules and a drawer processor of a (N−1)th one of the plurality of N drawer modules, and
an Nth one of the plurality of communication channels is electrically connected between the drawer processor of the (N−1)th one of the plurality of N drawer modules and a drawer processor of an Nth one of the plurality of N drawer modules.

17. The drawer system of claim 15, wherein the drawer processor of the second one of the plurality of N drawer modules is configured to assign the third address to a third one of the plurality of N drawer modules.

18. The drawer system of claim 15, wherein the drawer processor of the first one of the plurality of N drawer modules configured to assign a second address to the second one of the plurality of N drawer modules is configured to:
increment the first address of the first one of the plurality of N drawer modules; and
assign the incremented first address as the second address to the second one of the plurality of N drawer modules.

19. The drawer system of claim 17, wherein the drawer processor of the second one of the plurality of N drawer modules is configured to receive an acknowledgement from the third one of the plurality of N drawer modules that contains the third address.

20. The drawer system of claim 15, wherein at least one of the individual ones of the plurality of N drawer modules is divided to include two or more drawer housings.
